(19) United States
(12) Patent Application Publication
(10) Pub. No.: US 2004/0122059 A1
(43) Pub. Date: Jun. 24, 2004

(54) PPAR-gamma ligands in the treatment of asthma and allergies

(75) Inventors: Margherita T. Cantorna, State College, PA (US); Avery August, State College, PA (US); John P. Vanden Heuvel, Port Matilda, PA (US)

Correspondence Address: MORGAN LEWIS & BOCKIUS LLP, 1111 PENNSYLVANIA AVENUE NW, WASHINGTON, DC, 20004, US

(73) Assignee: The Penn State Research Foundation

(21) Appl. No.: 10/674,395

(22) Filed: Oct. 1, 2003

Related U.S. Application Data
(60) Provisional application No. 60/415,452, filed on Oct. 1, 2002.
(60) Provisional application No. 60/418,818, filed on Oct. 11, 2002.

(51) Int. Cl. A61K 31/4439; A61K 31/426; A61K 31/557

(52) U.S. Cl. 514/342; 514/369; 514/573

(57) ABSTRACT

Ligands for the nuclear hormone receptor PPARγ significantly reduced the immunological symptoms of allergic asthma in a murine model of this disease. In vitro, 15-deoxy-Delta(12,14)-prostaglandin J(2), a PPARγ ligand, significantly inhibited production of the T_{H}2 type cytokine IL-5 from T cells activated in vitro. More importantly, in a model of allergic asthma, mice treated orally with Ciglitazone had significantly reduced lung inflammation and mucous production following induction of allergic asthma. T cells from Ciglitazone treated mice also produced less IFNγ, IL-4 and IL-2 upon rechallenge in vitro with the model allergen. Our results suggest that ligands for PPARγ may be effective treatments for asthmatic patients.

### CROSS-REFERENCE TO RELATED APPLICATIONS

This application is claims benefit of U.S. Provisional Application No. 60/418,818, filed Oct. 11, 2002, and U.S. Provisional Application No. 60/415,452, filed Oct. 1, 2002, both of which are incorporated herein by reference in their entirety.

### STATEMENT REGARDING FEDERALLY SPONSORED RESEARCH OR DEVELOPMENT

This invention was made with U.S. government support under grant numbers PEN 02414 and PEN 03639 awarded by the U.S. Department of Agriculture. The U.S. government has certain rights in the invention.

### FIELD OF THE INVENTION

This invention is in the field of treatment of allergies, type I hypersensitivity and asthma. In particular, the invention relates to compounds that regulate PPARγ and the discovery that these compounds, PPARγ agonists, are effective in treating asthma, allergies and type I hypersensitivity.

### BACKGROUND OF THE INVENTION

Allergies reflect an overreaction of the immune system to substances that usually cause no reaction in most individuals. The symptoms include itching, sneezing, wheezing, and coughing. Allergies are estimated to affect nearly 50 million children and adults in the United States alone. The same substances that cause allergy symptoms can also trigger an asthma episode.

Asthma is a chronic inflammatory disease of the airways. Over 26 million Americans have asthma, 8.6 million of these people are under the age of 18. In children, asthma is the most common chronic childhood disease. Allergic asthma is thought to occur due to an aberrant immune response to airborne allergenic material.

In response to airborne allergens, the immune system of an asthmatic generates a strong type-2 helper T (T_{H}2) cell response in the lung airways. T_{H}2 cell cytokine production, inflammation, and eosinophil infiltration result in the increased production of mucous, and epithelial cell thickening which results in airway hyper-responsiveness (AHR). The T_{H}2 cell products interleukin (IL)-4, IL-5 and IL-13 have been associated with the disease severity in experimental allergic asthma. Introduction of antigen specific T_{H}2 cells alone, or IL-4 and IL-13 alone results in AHR, and blockade of these cytokines prevents the development of AHR in mice. In humans, blockade of the T_{H}2 cytokine IL-4 can relieve some of the symptoms of asthma. It seems likely that treatments which regulate T_{H}2 cell function might be used to ameliorate or modulate the symptoms of asthma.

The peroxisome proliferator activator receptors (“PPARs”) are members of the nuclear receptor superfamily. In the presence of PPAR ligands, the PPAR family regulates the transcription of targeted genes. The PPAR receptors were originally identified as regulators of adipocyte differentiation and lipid metabolism. Recently, PPAR-γ has been shown to be expressed in cells of the immune system including both T cells and macrophage. The endogenous ligands for PPAR-γ are thought to be lipids although there are also a number of synthetic drugs (the Thiazolidinediones Rosiglitazone, Ciglitazone and Troglitazone) that have been shown to regulate lipid and sugar metabolism via PPAR-γ.

The role of PPAR-γ as a regulator of the immune system is receiving a great deal of attention. PPARγ was observed to be highly expressed in myeloid cells and endogenous ligands such as 15-deoxy-Delta(12,14)-prostaglandin J(2) (PGJ2) as well as synthetic ligands regulated macrophage activation. In vitro PPAR-γ ligands have been shown to inhibit expression of inflammatory mediators including tumor necrosis factor-α and inducible nitric oxide synthase. In T cells, the PPARγ ligand Ciglitazone downregulates T cell proliferation and the production the T_{H}1 cytokines interferon (IFN)γ and IL-2 (Clark et al. 2000. J. Immun. 164: 1364; Cunard et al. 2002. J. Immun. 168: 2795). In vivo, PPAR-γ ligands suppressed symptoms of T_{H}1 driven diseases such as experimental autoimmune encephalomyelitis, type-1 diabetes, and colitis (Kawahito et al. 2000. J. Clin. Invest. 106: 189; Su et al. 1999. J. Clin. Invest. 104: 383; Niino et al. 2001. J. Neuroimmunol. 116: 40).

At present there is no cure for asthma, although there are a number of medications available to both relieve and prevent asthma attacks. Current pharmaceutical therapies available to asthma sufferers include the corticosteroids, mast stabilizers such as Cromolyn sodium and leukotriene modifiers. The immuno-suppressants cyclosporin A, FK506 and rapamycin, have also been suggested as likely treatments, although all these drugs non-specifically inhibit the immune system. In addition, long-term use of these drugs have been associated with side effects including osteoporosis, high blood pressure, and increased susceptibility to infection. More specific and new strategies for treating asthma depend on a better understanding of the disease process.

Thus, in view of the foregoing, there is a need in the art to obtain compounds and develop methods useful for the treatment of asthma, allergies and type I hypersensitive conditions.

### SUMMARY OF THE INVENTION

The invention is directed to a method for treating a subject having, or susceptible to having, a type I hypersensitivity, asthma or an allergy comprising administering a therapeutically effective amount of at least one PPAR-γ agonist, or derivative thereof, to said subject, wherein said administration of said at least one PPAR-γ agonist, or derivative thereof, is effective to treat said type I hypersensitivity, asthma or allergy in said subject.

In another aspect of the invention, the invention is directed to a method for treating a subject having, or susceptible to having, a type I hypersensitivity, asthma or allergy, comprising administering to said subject a therapeutically effective amount of a compound comprising Formula I:

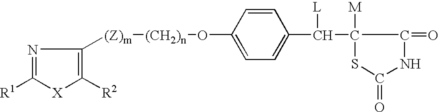

wherein R¹ is hydrogen, hydrocarbon residue, or heterocyclic residue which may each be substituted; R² is hydrogen or lower alkyl which may be substituted by a hydroxyl group; X is an oxygen or sulfur atom; Z is a hydroxylated methylene or carbonyl; m is a value of 0 or 1; n is an integer having a value of from 1 to 3; and L and M combine with each other and cooperate jointly to form a linkage and a plurality of salts.

In another aspect of the invention, the invention is directed to an in vivo method of identifying a compound effective to treat type I hypersensitivity, asthma or allergy in a subject comprising: a) contacting a group of one or more subjects with a test compound to form a first population; b) contacting a different group of one or more subjects with a PPAR-γ agonist to form a second population; c) inducing type I hypersensitivity, asthma or said allergy in said first and second populations; and, d) comparing one or more symptoms of said type I hypersensitivity, asthma or allergy in said first and second populations; wherein when said one or more symptoms of said type I hypersensitivity, asthma or allergy in said first population is less than or the same as said one or more symptoms of said type I hypersensitivity, asthma or allergy in said second population, a compound effective to treat type I hypersensitivity, asthma or allergy in a subject is identified. The invention is also directed to a compound identified by this in vivo method. In one embodiment, the invention is directed to the compound identified by the in vivo method in a pharmaceutically acceptable carrier.

In another aspect of the invention, the invention is directed to a method of regulating T_{H}2 cell function in the lung airway of a subject in need of said regulating comprising administering to said subject an amount of a PPAR-γ agonist effective to regulate said T_{H}2 cell function in said lung airway of said subject.

In yet a different aspect of the invention, the invention is directed to an in vitro method for identifying a compound effective to treat type I hypersensitivity, asthma or allergy in a subject comprising: a) culturing a first T cell population under T_{H}2 priming conditions to obtain a primed first cell population; b) culturing a second T cell population under T_{H}2 priming conditions to obtain a primed second cell population; c) stimulating said first primed cell population with a PPARγ agonist; d) stimulating said second primed cell population with said test compound; and, e) comparing the amount of secretion of one or more cytokines from said cell populations in part c) and part d); wherein when the cytokine secretion from said cell population of part d) is less than or equal to the cytokine secretion from the cell population of part c), a compound effective to treat type I hypersensitivity, asthma or allergy in a subject is identified. In one embodiment of this method, the PPARγ agonist is Ciglitazone. Also included in the invention is a compound identified by the in vitro method and the identified compound in a pharmaceutically acceptable or physiologically acceptable solution or carrier.

### BRIEF DESCRIPTION OF THE DRAWINGS

FIGS. 1A-B. Lungs from control mice. FIG. 1A. Histopathology score of 5 or Ciglitazone treated. FIG. 1B. Histopathology score of 1.

FIGS. 2A-F. Representative examples of PPAR-γ ligands. A. Rosiglitazone. B. Ciglitazone. C. Pioglitazone. D. Englitazone. E. Troglitazone. F. Prostaglandin J2.

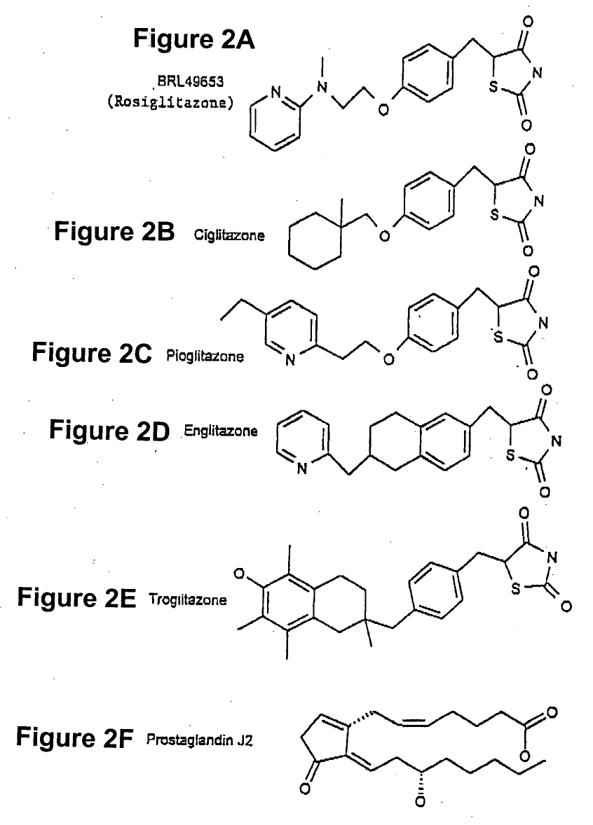

### DETAILED DESCRIPTION OF THE PREFERRED EMBODIMENTS

The invention is directed to a method for treating a subject having, or susceptible to having, a type I hypersensitivity, asthma or an allergy comprising administering a therapeutically effective amount of at least one PPAR-γ agonist, or derivative thereof, to said subject, wherein said administration of said at least one PPAR-γ agonist, or derivative thereof, is effective to treat said type I hypersensitivity, asthma or allergy in said subject.

In one aspect of the method of the invention, the PPAR-γ agonist is selected from the group consisting of a thiazolidinedione and a non-thiazolidinedione PPAR-γ agonist. In a preferred embodiment of the method, the at least one PPARγ agonist is selected from the group consisting of Ciglitazone, Troglitazone, Rosiglitazone, Pioglitazone, Englitazone, RXR activator LGD1069, and prostaglandin J2. In another preferred embodiment, the subject has, or is susceptible to having, an asthma. In a highly preferred embodiment, the asthma is allergic asthma.

In a different embodiment of the method, the therapeutically effective amount of said PPAR-γ agonist is approximately from 2 mg/kg to 10 mg/kg per day. In a more preferred embodiment, the therapeutically effective amount of said PPAR-γ agonist is approximately 2 mg/kg per day. In yet another embodiment of the method, the administering is selected from the group consisting of aerosol, parenteral, oral, intravenous, intramuscular, intraperitoneal, transdermal, rectal, buccal and subcutaneous administering. In a preferred embodiment, the subject is a mammal. In a highly preferred embodiment, the mammal is human.

In another aspect of the method, the PPAR-γ agonist is a thiazolidinedione derivative. In one embodiment, the thiazolidinedione derivative is administered by a route selected from the group consisting of aerosol, parenteral, oral, intravenous, intramuscular, intraperitoneal, transdermal, rectal, buccal and subcutaneous administration. In another embodiment, the thiazolidinedione derivative comprises a thiazolidinedone-2 derivative or a 4-diketone substituted derivative. In a preferred embodiment, the therapeutically effective amount of the thiazolidinedione derivative is approximately 2 mg/kg to 10 mg/kg per day. In another preferred embodiment, the therapeutically effective amount of the thiazolidinedione derivative is approximately 2 mg/kg per day. In a highly preferred embodiment, the subject is a mammal. In another highly preferred embodiment, the mammal is a human.

In another aspect of the method of the invention, the PPAR-γ agonist is a non-thiazolidinedione PPAR-γ agonist. In one embodiment, the non-thiazolidinedione PPAR-γ agonist is administered by a route selected from the group consisting of aerosol, parenteral, oral, intravenous, intramuscular, intraperitoneal, transdermal, rectal, buccal and subcutaneous administration. In a different embodiment of the method, the non-thiazolidinedione PPAR-γ agonist comprises a piperazine or heterocycle derivative. In a preferred embodiment, the therapeutically effective amount of said non-thiazolidinedione PPAR-γ agonist is approximately 2 mg/kg to 10 mg/kg per day. In a highly preferred embodiment, the therapeutically effective amount of said non-thiazolidinedione PPAR-γ agonist is approximately 2 mg/kg per day. In another highly preferred embodiment, the subject is a mammal. In a very highly preferred embodiment, the mammal is a human.

In another aspect of the invention, the invention is directed to a method for treating a subject having, or susceptible to having, a type I hypersensitivity, asthma or allergy, comprising administering to said subject a therapeutically effective amount of a compound comprising Formula I:

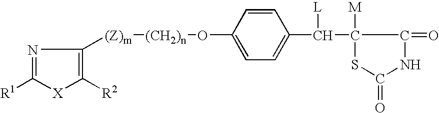

wherein R¹ is hydrogen, hydrocarbon residue, or heterocyclic residue which may each be substituted; R² is hydrogen or lower alkyl which may be substituted by a hydroxyl group; X is an oxygen or sulfur atom; Z is a hydroxylated methylene or carbonyl; m is a value of 0 or 1; n is an integer having a value of from 1 to 3; and L and M combine with each other and cooperate jointly to form a linkage and a plurality of salts. In one aspect of this method, the subject has, or is susceptible to having, an asthma. In a highly preferred embodiment, the asthma is allergic asthma. In a different aspect of the method, the therapeutically effective amount of said compound is approximately from 2 mg/kg to 10 mg/kg per day. In a preferred embodiment, the therapeutically effective amount of said PPAR-γ agonist is approximately 2 mg/kg per day.

In one embodiment, the administering is selected from the group consisting of aerosol, parenteral, oral, intravenous, intramuscular, intraperitoneal, transdermal, rectal, buccal, or subcutaneous administration. In a preferred aspect, the subject is a mammal. In a highly preferred aspect, the mammal is a human.

In another aspect of the invention, the invention is directed to an in vivo method of identifying a compound effective to treat type I hypersensitivity, asthma or allergy in a subject comprising: a) contacting a group of one or more subjects with a test compound to form a first population; b) contacting a different group of one or more subjects with a PPAR-γ agonist to form a second population; c) inducing type I hypersensitivity, asthma or said allergy in said first and second populations; and, d) comparing one or more symptoms of said type I hypersensitivity, asthma or allergy in said first and second populations; wherein when said one or more symptoms of said type I hypersensitivity, asthma or allergy in said first population is less than or the same as said one or more symptoms of said type I hypersensitivity, asthma or allergy in said second population, a compound effective to treat type I hypersensitivity, asthma or allergy in a subject is identified. In one embodiment, the one or more symptoms is selected from the group consisting of an increase in T_{H}2 type cytokines, lung airway inflammation, eosinophil infiltration, mucous production in the lung, airway hyperreactivity (AHR) and elevated serum IgE levels. In another embodiment, the subject is a mammal. In a preferred embodiment, the mammal is a human. In a highly preferred embodiment, the asthma is allergic asthma. The invention is also directed to a compound identified by this in vivo method. In one embodiment, the invention is directed to the compound identified by the in vivo method in a pharmaceutically acceptable carrier. In one embodiment of the in vivo method the agonist is Ciglitazone.

In another aspect of the invention, the invention is directed to a method of regulating T_{H}2 cell function in the lung airway of a subject in need of said regulating comprising administering to said subject an amount of a PPAR-γ agonist effective to regulate said T_{H}2 cell function in said lung airway of said subject. In one embodiment, the T_{H}2 cell function is selected from the group consisting of T_{H}2 cell cytokine production, inflammation, eosinophil infiltration, mucous production, airway hyperreactivity (AHR) and epithelial cell thickening. In another embodiment, the T_{H}2 cell cytokine production comprises production of IL-4, IL-5 and IL-13.

In yet a different aspect of the invention, the invention is directed to an in vitro method for identifying a compound effective to treat type I hypersensitivity, asthma or allergy in a subject comprising: a) culturing a first T cell population under TH² priming conditions to obtain a primed first cell population; b) culturing a second T cell population under T_{H}2 priming conditions to obtain a primed second cell population; c) stimulating said first primed cell population with a PPARγ agonist; d) stimulating said second primed cell population with said test compound; and, e) comparing the amount of secretion of one or more cytokines from said cell populations in part c) and part d); wherein when the cytokine secretion from said cell population of part d) is less than or equal to the cytokine secretion from the cell population of part c), a compound effective to treat type I hypersensitivity, asthma or allergy in a subject is identified. In one embodiment of this method, the PPARγ agonist is Ciglitazone. In a different embodiment, the one or more cytokines is selected from the group consisting of IL-2, IL-5, IL-13 and IFNγ. In a preferred embodiment of the invention, the subject is a mammal. In a highly preferred embodiment, the mammal is human. Also included in the invention is a compound identified by the in vitro method and the identified compound in a pharmaceutically acceptable or physiologically acceptable solution or carrier.

### DEFINITIONS

As is generally the case in biotechnology and chemistry, the description of the present invention has required the use of a number of terms of art. Although it is not practical to do so exhaustively, definitions for some of these terms are provided here for ease of reference. Unless defined otherwise, all technical and scientific terms used herein have the same meaning as commonly understood by one of ordinary skill in the art to which this invention belongs. Definitions for other terms also appear elsewhere herein. However, the definitions provided here and elsewhere herein should always be considered in determining the intended scope and meaning of the defined terms. Although any methods and materials similar or equivalent to those described herein can be used in the practice of the present invention, the preferred methods and materials are described.

As used herein the term “agonist” refers to compounds that exert their effects by interacting with a receptor in the same fashion as the endogenous ligand for the receptor, thereby mimicking the action of the endogenous molecule. In terms of the present patent application, the receptor is PPAR-γ. Agonists of this receptor include thiazolidinediones such as, for example, Ciglitazone, Pioglitazone and Rosiglitazone. The endogenous ligand is somewhat controversial but includes compounds such as, for example, Delta12,14-prostaglandin J2 (PGJ₂), 9-S-hydroxyoctadecadienoic acid (9-S-HODE), and 13-S-HODE. Agonists of PPAR-γ result in activation of this receptor and a variety of beneficial effects, including reversing the symptoms of asthma.

As used herein, the term “activation” refers to “transcriptional activation.” In other words, activating PPAR-γ would signify an increase in the ability of this receptor to increase or decrease gene expression. When PPAR-γ is activated, there is a decrease in symptoms of asthma through regulation of a particular subset of genes.

As used herein, the term “antagonist” refers to compounds that have actions which are the opposite of those of an endogenous compound or agonist. There have been a few PPAR-γ antagonists described, such as, for example, GW9662.

As used herein, the term “asthma” refers to a chronic, inflammatory lung disease characterized by recurrent breathing problems. People with asthma have acute episodes when the air passages in their lungs get narrower, and breathing becomes more difficult. Sometimes episodes of asthma are triggered by allergens, although infection, exercise, cold air and other factors are also important triggers. Allergic asthma, also known as extrinsic asthma, is asthma that is triggered by an allergic reaction, usually something that is inhaled. Intrinsic asthma is asthma that has no apparent external cause.

As used herein, the term “derivative thereof” refers to a chemically modified compound wherein the chemical modification takes place at one or more functional groups of the agent and/or on an aromatic ring, when present. The derivative however is expected to retain the pharmacological activity of the agent from which it is derived.

As used herein, the term “PPAR-γ agonist” includes, but is not limited to, the compounds of Formula I and derivatives thereof. Unless stated otherwise, the term includes the phrase “selective PPAR-γ agonist.”

As used herein, the term “prodrug” refers to a compound which is convertible in vivo by metabolic means, such as, for example, hydrolysis, to a compound of Formula (I). For example an ester of a compound of Formula (I) or of a PPAR-γ agonist containing a hydroxy group may be convertible by hydrolysis in vivo to the parent molecule. Alternatively an ester of a compound of Formula (I) or of a PPAR-γ agonist containing a carboxy group may be convertible by hydrolysis in vivo to the parent molecule.

As used herein, the term “susceptible” refers to the predisposition or liklihood that a particular subject will develop an allergy, asthma or type I hypersensitivity. Such subjects do not exhibit detectable symptoms of an existing allergy, asthma or type I hypersensitivity. The allergy, asthma or type I hypersensitivity may not have yet developed, is inactive, or has not progressed to the point where symptoms or indications are exhibited by the subject.

As used herein, the term “therapeutically effective amount” refers to an amount of drug or pharmaceutical agent that will elicit the biological or medical response of a tissue, system, animal, or human that is being sought. In particular, the term refers to a symptom-ameliorating effective amount and, in the case of subjects with mild or moderate symptoms, an amount effective to inhibit progression to more severe symptoms. See, for example, U.S. Pat. No. 6,462,046.

Ligands

Many PPAR-γ ligands are known. PPAR-γ ligands which are approved for human-use drugs are Rosiglitazone (Avandia, GlaxoSmithKline) and Pioglitazone (Actos, Lilly). PPAR-γ ligands which are not approved for human use are Englitazone (Pfizer), Ciglitazone (Upjohn), and, Troglitazone (Rezulin, Parke-Davis; taken off the market).

PPAR-γ Agonists

Preferred compounds for the practice of the present invention include compounds which bind to the PPARγ receptor and are effective in lessening the symptoms of allergy, Type 1 hypersensitivity and asthma. In order to identify or evaluate, either in vitro or in vivo, whether a candidate compound is suitable for the present invention, one would test the compound side-by-side with Ciglitazone and determine the effective dose of the compound in alleviating, for example, allergic asthma symptoms (as described here). A successful compound would be one that was at least as effective as Ciglitazone or better than Ciglitazone for the treatment of allergic asthma. The method may be used, in the same manner, to identify compounds effective in the treatment of type I hypersensitivity or an allergy. The present invention demonstrates that agents that regulate PPAR-γ (e.g., PPAR-γ agonists) are effective at inhibiting experimentally induced allergic asthma. Preferred compounds include, but are not limited to, Ciglitazone, Troglitazone, Rosiglitazone, Pioglitazone, RXR activator LGD 1069, prostaglandin J2 and, derivatives thereof.

Additional compounds useful for practicing the present invention, and methods of making these compounds are known. Some of these compounds are disclosed in WO 91/07107; WO 94/01433; WO 92/02520; WO 89/08651; JP Kokai 69383/92; U.S. Pat. Nos. 4,287,200; 4,340,605; 4,438,141; 4,444,779; 4,461,902; 4,572,912; 4,687,777; 4,703,052; 4,725,610; 4,873,255; 4,873,393; 4,897,405; 4,918,091; 4,948,900; 5,02,953; 5,061,717; 5,120,754; 5,132,317; 5,194,443; 5,223,522; 5,232,925; 5,260,445; 6,462,046; 6,437,143; 6,214,850; 6,191,154; 6,605,627, and 6,596,693. The active compounds described therein and methods of making and using the active compounds referenced in these publications are included herein and are within the scope of the present invention.

Compounds effective for the treatment of asthma, allergy and type I hypersensitivity include PPAR-γ agonists, such as the thiazolidinediones, and their derivatives, of general formula I:

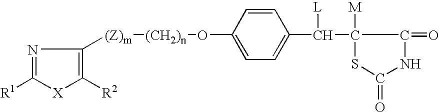

Wherein R¹ is hydrogen or a hydrocarbon residue or heterocyclic residue which may each be substituted; R² is hydrogen or lower alkyl which may be substituted by hydroxyl group; X is an oxygen or sulfur atom; Z is a hydroxylated methylene or carbonyl; m is 0 or 1; n is an integer of 1 to 3; L and M combine with each other to cooperate jointly to form a linkage and their salts.

Other thiazolidinediones, and their derivatives, are possible. See, for example, U.S. Pat. No. 6,437,143. Non-thiazolidinedione PPARγ agonists have also been described. See, for example, piperazine or heterocycle derivatives described in U.S. Pat. No. 6,462,046.

Certain of the PPAR-γ agonists may exist as optical isomers and the invention includes the racemates, racemic mixtures, as well as the individual entantiomers or diastereomers and all isomeric forms as well as mixtures thereof. Isomers, racemates, enantiomers and diastereomers may be separated according to methods well known to those of ordinary skill in the art and are included in the present invention as well as mixtures thereof. See, for example, U.S. Pat. Nos. 6,462,046 and 6,437,143.

The PPAR-γ selective agonists contemplated for use in this invention are used in both in vitro and in vivo applications. For in vivo applications, the agonists are incorporated into a pharmaceutically acceptable formulation for administration and administered by methods known in the art. For in vitro applications, the agonists may be incorporated into physiologically acceptable formulations for use or in formulations other than physiologically acceptable formulations which are suitable for the intended use thereof.

In Vitro Assays

In vitro assays are included within the scope of the invention. Provided herein are methods for identifying compounds effective to treat type I hypersensitivity, asthma or allergy in a subject in need of such treatment. These methods include direct binding assays and reporter assays. See, for example, DeGrazia et al. 2003. Bioorg. Med. Chem., In Press; Yu et al. 2002. Biochim. Biophys. Acta. 1581: 89; and, Mueller et al., 2003.

Also included within the scope of the present invention are the non-toxic pharmaceutically acceptable salts of the PPAR-γ selective agonists of the present invention. Acidic salts are formed by mixing a solution of the agonists with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Examples of other pharmaceutically acceptable salts include salts such as hydrobromide, sulphate, lactate, mandelate, and oxalate. See, for example, U.S. Pat. No. 6,613,803.

Basic salts are formed by mixing a solution of PPAR-γ selective agonists with a solution of a pharmaceutically acceptable non-toxic base such as sodium hydroxide, potassium hydroxide, choline hydroxide, sodium carbonate and the like. Examples of basic salts of the agonists include the pharmacologically acceptable salts, such as the sodium salts, potassium salts, magnesium salts, calcium salts, amine salts and other salts of the same type (aluminum, iron, bismuth and the like). The amine salts which are not pharmacologically acceptable may serve as means for identification, purification or resolution. See, for example, U.S. Pat. No. 6,437,143.

Also included in the scope of the invention are pharmaceutically acceptable salts of the agonists, where a basic or acidic group is present in the structure. When an acidic substituent is present, the ammonium, morpholinium, sodium, potassium, barium, calcium salt, for example, can be formed for use as the dosage form. When a basic group such as, for example, an amino or a basic heteroaryl radical, such as pyridyl, is present, an acidic salt, such as for example, hydrochloride, hydrobromide, acetate, oxalate, maleate, fumarate, citrate, palmoate, methanesulfonate, p-toluenesulfonate, can be formed as the dosage form. See, for example, U.S. Pat. No. 6,462,046.

In addition, some of the agonists of the instant invention may form solvates with water or common organic solvents, and those solvates are encompassed within the scope of the invention. See, for example, U.S. Pat. No. 6,462,046.

Also, where acidic groups are present, pharmaceutically acceptable esters can be employed, such as, for example, methyl, tert-butyl, pivaloyloxymethyl, and those esters known in the art for modifying solubility or hydrolysis characteristics for use as sustained release or prodrug formulations. Examples of prodrugs include esters or amides of the agonists and these may be prepared by reacting compounds, such as those of Formula I and other PPAR-γ agonists, for example, with anhydrides. Such methods are well known and available to those of skill in the art.

While individual needs vary, determination of optimal ranges of effective amounts of PPAR-γ selective agonists is within the skill of the art. Compositions within the scope of this invention include all compositions wherein the PPAR-γ selective agonists of the present invention are contained in an amount which is effective to achieve their intended purpose. Typically, the PPAR-γ selective agonists may be administered to subject such as mammals, particularly, humans, for example, orally at a dose of 0.0025 to 50 mg/kg, or an equivalent amount of the pharmaceutically acceptable salt thereof. PPAR-γ selective agonists compounds can be administrated by intravenous injection at a dose of about 0.025 to about 10 mg/kg.

An oral dose of the PPAR-γ selective agonists may comprise from about 0.01 to about 50 mg, preferably about 0.1 to about 10 mg of the compounds. The oral dose may be administered one or more times daily as one or more tablets each containing from about 0.1 to about 10, conveniently about 0.25 to 50 mg of the PPAR-γ selective agonist or its solvates. As a general rule, the dosage can range from 1 to 200 mg per dose and the daily dosage can range from 2 to 500 mg. See, for example, U.S. Pat. No. 6,437,143.

Ultimately, the dosage administered will be dependent upon a number of factors including the activity of the particular compound being administered, the age, body weight, general health, sex, diet, time of administration, route of administration, rate of excretion, drug combination, kind of concurrent treatment, frequency of treatment, nature of the effect desired, and, the severity of the particular disease undergoing therapy. See, for example, Remington's Pharmaceutical Sciences (2000) Mack Publishing Company, Easton, Pa., USA; The Physician's Desk Reference (PDR) 2003; and, Goodman & Gilman's The Pharmacological Basis of Therapeutics (2001) By Louis Sanford Goodman, Alfred G. Gilman, Lee E. Limbird, Joel G. Hardman, Alfred Goodman Gilman, McGraw-Hill.

When administered via aerosol, the PPARγ agonist is present in a therapeutically effective amount, that is, in an amount such that the PPARγ agonists can be administered as a dispersion or an aerosol typically preferred with one dose, or through several doses. Reproducible dosing can be achieved by means known to those of skill in the art. The final aerosol formulation will contain that amount of active compound effective to give the results intended. See, for example, U.S. Pat. Nos. 6,613,307; 6,610,272; and, 6,596,261.

Administration Forms

The pharmaceutical PPAR-γ selective agonists of the present invention may be administered by any means that achieve their intended purpose. For example, administration may be by one or more of the following routes: aerosol, parenteral, subcutaneous, intravenous, intramuscular, intraperitoneal, transdermal, buccal, oral and rectal routes, either alternatively or concurrently, at a single administration or at multiple administrations over any length of time necessary to achieve the intended result.

In addition to administering the PPAR-γ selective agonists as a single compound, the agonists of the invention may be administered as part of a pharmaceutical preparation containing suitable pharmaceutically acceptable carriers comprising excipients and auxiliaries which facilitate processing of the compounds into preparations which can be used pharmaceutically. Oral preparations, such as tablets, dragees, and capsules, and also preparations which can be administered rectally, such as suppositories, as well as suitable solutions for administration by injection or orally, contain from about 0.01 to 99 percent, preferably from about 0.25 to 75 percent of active PPAR-γ selective agonists, together with the excipient. See, for example, U.S. Pat. Nos. 6,596,693; 6,437,143 and 6,462,046.

The pharmaceutical compositions of the invention may be administered to any subject in need of the beneficial effects of the PPAR-γ selective agonists. Such subjects include, but are not limited to, mammals, and particularly, humans.

The pharmaceutical preparations comprising PPAR-γ agonists are manufactured by any means known, such as for example, by mixing, granulating, dissolving, or lyophilizing processes. Thus, pharmaceutical preparations of PPARγ selective agonists for oral use can be obtained by combining the active compounds with solid excipients, to obtain tablets, capsules or dragee cores. Tablets may contain pharmaceutically acceptable excipients as an aid in the manufacture of such tablets. As is conventional in the art, these tablets may be coated with a pharmaceutically acceptable enteric coating, such as glyceryl monostearate or glyceryl distearate, to delay disintegration and absorption in the gastrointestinal tract to provide a sustained action over a longer period.

Suitable excipients can be, for example, fillers such as saccharides, lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone.

If desired, disintegrating agents may be added such as starches and also carboxymethyl-starch, cross-linked polyvinyl pyrrolidone, agar, or alginic acid or a salt thereof, such as sodium alginate. Auxiliaries are flow-regulating agents and lubricants, and include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used.

The compounds of the present invention may be administered orally as oily or aqueous suspensions, lozenges, troches, powders, granules, emulsions, syrups or elixars. Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

The compositions for oral use may include at least one agent for flavoring, sweetening, coloring and preserving in order to produce pharmaceutically acceptable preparations. Dye stuffs or pigments may be added to the tablets or dragee coatings, for example, for identification or to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases include, for example, natural or synthetic triglycerides, paraffin hydrocarbons, liquid triglycerides, polyethylene glycols, or paraffin hydrocarbons. These compositions can be prepared by mixing the compounds with a suitable non-irritating excipient that is solid at about room temperature but liquid at rectal temperature and will therefor melt in the rectum to release the compounds. Examples of such materials are cocoa butter and other glycerides.

Suitable formulations for parenteral administration include aqueous solutions of the active compounds in water-soluble form, for example, water-soluble salts and alkaline solutions. The sterile injectable preparation may also be formulated as a suspension in a non toxic parenterally-acceptable diluent or solvent, for example as a solution in 1,3-butanediol. Among the acceptable vehicles and solvents that may be employed are water, Ringers solution and isotonic sodium chloride solution.

Suspensions of the active compounds as oily injection suspensions may be administered. Suitable lipophilic solvents or vehicles include polyethylene glycol-400, fatty oils, sesame oil, and synthetic fatty acid esters, such as for example, ethyl oleate or triglycerides, and, synthetic mono- or diglycerides.

Aqueous injection suspensions may contain substances which increase the viscosity of the suspension, and include, for example, sodium carboxymethyl cellulose, sorbitol, and/or dextran. Optionally, the suspension may also contain stabilizers. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occuring phosphatide such as for example, lecithin, polyoxyethylene stearate, heptadecaethylen-oxycetanol, polyoxyethylene sorbitol monooleate or fatty acid hexitol anhydrides such as polyoxyethylene sorbitan monooleate.

For topical use preparations, for example, creams, ointments, jellies solutions, or suspensions, containing the compounds of the present invention are employed.

The compounds of the present invention may also be administered in the form of liposome delivery systems such as small unilamellar vesicles, large unilamellar vesicles and multimellar vesicles. Liposomes can be formed from a variety of phospholipids, such as cholesterol, stearylamine or phosphatidylcholines. Methods of forming liposomes are known to those of skill in the art.

Delivery of therapeutic agents to the lung by way of inhalation is an important means of treating asthma and chronic obstructive pulmonary disease. Steroids, beta-agonists, anti-cholinergic agents, proteins and polypeptides are among the therapeutic agents that are administered to the lung for such purposes. The invention herein involves a method of treatment using an aerosol formulation which comprises (a) one or more PPARγ agonists; and (b) a suitable fluid carrier. See, for example, U.S. Pat. Nos. 6,613,307; 6,610,272; and, 6,596,261.

Generally, the PPARγ agonist formulations of the invention can be prepared by combining (a) PPARγ agonists in an amount sufficient to provide a plurality of therapeutically effective doses; (b) the propellant, in an amount sufficient to propel a plurality of doses from an aerosol canister; (c) optionally, the water addition in an amount effective to further stabilize each of the formulations; and (d) any further optional components, such as, for example, ethanol as a cosolvent; and dispersing the components. The components can be dispersed using a conventional mixer or homogenizer, by shaking, or by ultrasonic energy as well as by the use of a bead mill or a microfluidizer. Bulk formulations can be transferred to smaller individual aerosol vials by using valve to valve transfer methods, pressure filling or by using conventional cold-fill methods. See, for example, U.S. Pat. Nos. 6,613,307; 6,610,272; and, 6,596,261.

It is not required that a component used in a suspension aerosol formulation be soluble in the fluid carrier, such as a propellant. Components that are not sufficiently soluble can be coated or congealed with polymeric, dissolution controlling agents in an appropriate amount and the coated particles can then be incorporated in a formulation as described above. Polymeric dissolution controlling agents suitable for use in this invention include, but not limited to polylactide glycolide co-polymer, acrylic esters, polyamidoamines, substituted or unsubstituted cellulose derivatives, and other naturally derived carbohydrate and polysaccharide products such as zein and chitosan. See, for example, U.S. Pat. Nos. 6,613,307; 6,610,272; and, 6,596,261.

Therapeutic agents are commonly administered to the lung in the form of an aerosol of particles of respirable size (less than about 10μ in diameter). The aerosol PPARγ agonist formulation can be presented as a liquid or a dry powder. In order to assure proper particle size in a liquid aerosol, particles can be prepared in a size suitable for respiration and then incorporated into a colloidial dispersion either containing a propellant as a metered dose inhaler (MDI) or air, such as in the case of a dry powder inhaler (DPI). Alternatively, the PPARγ agonist formulations can be prepared in solution form in order to avoid the concern for proper particle size in the formulation. Solution formulations of PPARγ agonists must nevertheless be dispensed in a manner that produces particles or droplets of respirable size. For MDI application, once prepared, an aerosol formulation is filled into an aerosol canister equipped with a metered dose valve. See, for example, U.S. Pat. Nos. 6,613,307; 6,610,272; and, 6,596,261.

For purposes of the methods of administration, the PPARγ agonists can also be micronized whereby a therapeutically effective amount or fraction of the PPARγ agonist is particulate. Typically, the particles have a diameter of less than about 10 microns, and preferably less than about 5 microns, in order that the particles can be inhaled into the respiratory tract and/or lungs.

A number of medicinal aerosol formulations using propellant systems are disclosed in, for example, U.S. Pat. No. 6,613,307 and the references cited therein (such as, for example, EP 0372777, WO91/04011, WO91/11173. WO91/11495, WO91/14422, WO92/00107, WO93/08447, WO93/08446. WO93/11743, WO93/11744 and WO93/11745) all of which are incorporated by reference herein in their entirety. Many such propellants are known in the art and are suitable for use in the invention herein. The propellants for use in the invention may be any fluorocarbon, hydrogen-containing fluorocarbon or hydrogen-containing chlorofluorocarbon propellant or mixtures thereof having a sufficient vapour pressure to render them effective as propellants. Suitable propellants include, for example, chlorofluorocarbons. The propellant may additionally contain a volatile adjuvant such as a saturated hydrocarbon for example propane, n-butane, isobutane, pentane and isopentane or a dialkyl ether for example dimethyl ether.

Where a surfactant is employed in the aerosol, it is selected from those which are physiologically acceptable upon administration by inhalation such as oleic acid, sorbitan trioleate (Span R 85), sorbitan mono-oleate, sorbitan monolaurate, polyoxyethylene (20) sorbitan monolaurate, polyoxyethylene (20) sorbitan monooleate, natural lecithin, fluorinated and perfluorinated surfactants including fluorinated lecithins, fluorinated phosphatidylcholines, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil and sunflower seed oil. See, for example, U.S. Pat. No. 6,613,307.

Also provided herein for use in the methods are aerosol formulations which contain PPARγ agonists and additionally one or more therapeutic agents. The additional therapeutic agents may be selected from any other suitable drug useful in inhalation therapy and which may be presented in a form which is substantially completely insoluble in the selected propellant. Appropriate medicaments may thus be selected from, for example, salbutamol, fluticasone propionate or beclomethasone dipropionate; analgesics, such as codeine, antiinfectives such as the cephalosporins and penicillins; antihistamines, such as for example, methapyrilene; anti-inflammatories, such as for example flunisolide, budesonide, tipredane or triamcinolone acetonide; antitussives,such as for example, noscapine; bronchodilators, such as for example, ephedrine, adrenaline, fenoterol, formoterol, isoprenaline, metaproterenol, phenylephrine,phenylpropanolamine, pirbuterol, reproterol, rimiterol, terbutaline, isoetharine, tulobuterol, (-)-4-amino-3,5-dichloro-.alpha.-[[[6-[2-(2-pyridinyl)ethoxy]hexyl]amino]m ethyl]benzenemethanol or orciprenaline; hormones, such as for example cortisone, hydrocortisone or prednisolone; xanthines such as for example aminophylline, choline theophyllinate, lysine theophyllinate or theophylline; and therapeutic proteins and peptides. Where appropriate, the PPARγ agonists may be used in the form of salts, esters or as solvates to optimize the activity and/or stability of the PPARγ agonists and/or to minimize the solubility of the PPARγ agonists in the propellant. See, for example, U.S. Pat. No. 6,613,307.

Animal Models

Airway hyperresponsiveness and inflammation are important features of human asthma. Mouse models are powerful tools for analyzing asthma and other genetically complex diseases. See, for example, Zhang et al. (Zhang et al., Human Mol. Gen. 1999. 8(4): 601).

Types of Diseases Treatable with the Compounds of the Invention

Other diseases or conditions treatable with the agonists of the invention include T_{H}2 mediated diseases, such as Allergic Rhinitis (hay fever), Atopic Eczema (AE) and some food allergies. T_{H}2 mediated immune responses mediated by the secretion of IL-4, IL-5 and IL-13 are important in the pathogenesis of atopic disorders, such as for example, allergen-induced asthma, rhinoconjunctivitis and anaphylaxis (Lewis, D. Oct. 2002. Curr. Opin. Immunol. 14(5): 644). The concept of AE (Atopic Eczema) starting with T_{H}2 inflammation, becoming T_{H}1 inflammation in chronicity, and finally progressing to an autoimmune disease with IgE antibodies against autologous epidermal proteins is very attractive (Ring et al. Jan. 2001. Curr. Allergy Rep. 1(1): 39)

The present inventors have examined whether by regulating specific cytokine production in T cells, ligands for PPAR-γ can regulate the development of the T_{H}2 mediated disease, allergic asthma, in a murine model. It is shown here that the PPAR-γ ligand 15-deoxy-delta-prostaglandin J₂ (PJG2) significantly inhibits antigen stimulated T cell production of the T_{H}2 type cytokine IL-5 in vitro. The inventors also show that treatment of allergen-primed mice with Ciglitazone significantly reduces the lung inflammation and mucous production following induction of allergic asthma using the model antigen ovalbumin(OVA). Furthermore, it is demonstrated that OVA specific T cells from the Ciglitazone treated mice proliferated less, produced less IL-2, IFN-γ, and IL-4 than control treated animals. It is contemplated that Ciglitazone and other PPARγ ligands are useful drugs for the treatment of asthma, type I hypersensitivity and other allergic diseases.

The following examples are illustrative, but not limiting, of the method and compositions of the present invention. Other suitable modifications and adaptations of the variety of conditions and parameters normally encountered in clinical therapy and experimental use and which are obvious to those skilled in the art are within the spirit and scope of the invention.

### EXAMPLES

### Material and Methods

Animals and diets. Groups of age and sex matched Balb/c (Jackson Labs, Bar Harbor, Me.) mice were used for experiments at 9-11 weeks of age. All mice were fed synthetic diets as described (Cantorna et al. 2000. J. Nutr. 130: 2648). T cell receptor (TCR) transgenic mice (D011.10 specific for ovalbumin, OVA) were also obtained from Jackson Laboratories. TCR transgenic mice were chosen because they provide a good source of CD4+T cells, which are 90% transgenic with known antigenic specificity.

T_{H} cell cultures. Lymph node and spleen cells from the DO11.10 TCR transgenic mice were removed and CD4 positive cells were isolated using the CD4 Immunocolumns and the manufacturer's instructions (Cedarlane labs, Homby, Canada). CD4 positive T cells (95-98% pure) were cultured in vitro with OVA and mitomycin C treated splenocytes from congenic mice. The cultures were incubated under neutral conditions (T_{H}0 priming) or with rIL-4 and anti-IFN-γ antibodies (T_{H}2 priming), or with rIL-12 (T_{H}1 priming). After 4 days (4d) in culture the cells were collected and dead splenocytes were removed by centrifugation over histopaque. At this time flow cytometry revealed that 95-98% of the remaining cells were transgenic CD4+T cells (using antibody KJ1-26, which identifies D011.10 transgenic T cells, Caltag, Burlingame, Calif.). T cells were collected and recultured with rIL-2 (Pharmingen) for 3 d to expand the T cells. Cells were then collected, washed and stimulated for proliferation and cytokine synthesis with phorbal myristiyl acetate (PMA, Sigma, St. Louis, Mo.) and ionomycin (Sigma) in the presence of PJG2, conjugated linoleic acid (CLA) or control treated.

Measurement of lymphocyte proliferation and cytokine production. Th cells were cultured in 96 well plates for proliferation assays and 24 well plates for supernatants (Corning Costar, Corning, N.Y.). After 72 h, supernatants were collected for ELISAs. For proliferation assays, 0.4 μCi of [3H]thymidine (ICN, Costa Mesa, Calif.) was added to each well and the cells were incubated for an additional 18 h. Radioactive thymidine incorporation was determined by liquid scintillation using a Beta plate Counter. Mouse IL-2, IL-4, IL-5 and IFN-γ production were detected using ELISA kits from Pharmingen, following the instructions provided. The ELISA detection limits were 25 pg/ml IL-2, 62 pg/ml IL-4, 312 pg/ml IL-5, and 1000 pg/ml IFN-γ.

Asthma induction and Ciglitazone treatment. Asthma was induced in the mice as described (Zhang et al. 1999. Immunity 11: 473). Briefly mice were injected intraperitoneally with 10 82 g of OVA in Alum twice on day 1 and 5 of asthma induction (primed). On day 10, mice were challenged intranasally with OVA in sterile saline, every day for three days (40 μg total). In an alternative protocol the mice were only challenged intranasally (unprimed) with OVA for three consecutive days. Asthma induction was effective with or without the use of immunization (Alum and OVA injections).

Two weeks after asthma induction (the day after the last intranasal challenge) the mice were sacrificed. Two different Ciglitazone treatments were done. In the first experimental design the mice were started on diets, which included 0.12 μM Ciglitazone/daily per mouse or control mice fed the same diets without Ciglitazone for one week prior to the induction of asthma. The mice continued on these diets for the remainder of the study. In the second experimental design the mice were fed control diets during the initial stages of asthma induction (immunization) and changed to diets, which included 0.12 μM Ciglitazone/daily per mouse on day 8, just prior to the intranasal exposure (late Ciglitazone treatment). Mice were fed 4 g of diet daily in order to insure that each mouse received its daily dose of Ciglitazone and that controls did not eat more than the Ciglitazone treated mice.

Asthma severity. The lungs were fixed in formalin and sent to the Penn State University Animal Diagnostic Laboratories for sectioning and staining with hematoxylin and eosin. The sections were scored blindly on a scale of 0 to 3 for inflammation and 0-3 for epithelial thickening. Inflammation: 0—no inflammation, 1—inflammatory cells present, 2—multiple loci of inflammation, 3—many inflammatory cells throughout (granulomas). Epithelial thickening: 0—normal, 1—some epithelial thickening, 2—multiple loci of thickened/obstructed airway, 3—normal structure not present, little to no unobstructed airway. The inflammation and epithelial scores for each section were added together for a total histopathology score ranging from 0-6. The results are presented as means±SEMs.

Microarray Analysis

The Mouse Genome Oligo Set Version 1 (Operon, Alameda, Calif.) contains 6800 optimized 70-mer plus 24 controls. Oligonucleotides were printed onto glass slides using GeneMachines Omnigrid (San Carlos, Calif.) with additional controls obtained from Stratagene (SpotReport system, La Jolla, Calif.) at the Penn State University microarray core facility. RNA was isolated from the lungs previously flash-frozen from five unprimed mice on control diets and five unprimed mice on the Ciglitazone diets. Total RNA was isolated by TriReagent (Sigma) and purified further with RNAEasy (Qiagen) according to the manufacturers' instructions. Slides were hybridized with cDNA labeled with Cy3 or Cy5 dye (Amersham) for 20-24 h at 42° C., washed, and immediately scanned using a GenePix 4000A scanner (Axon Instruments, Foster City, Calif.) with dedicated PC running Axon GenePix image acquisition and analysis software. Analysis of gene expression was performed using GeneSpring software (SiliconGenetics, Redwood City, Calif).

Normalization and analysis of the gene expression profiles were performed as follows: some genes were used as negative controls for a background subtraction; their median value was subtracted from the raw values for each gene before anything else was done. Each gene's measured intensity was divided by its control channel value in each sample. When the control channel value was below 10.0, the data point was considered bad. Intensity-dependent normalization was also applied, where the ratio was reduced to the residual of the lowest fit of the intensity vs. ratio curve. The 50th percentile of all measurements was used as a positive control for each sample; each measurement for each gene was divided by this synthetic positive control, assuming that this was at least 0.01. The bottom 10th percentile was used as a test for correct background subtraction. This was never less than the negative of the synthetic positive control.

The values reported are the ratio of the median intensities of red fluorescence over green fluorescence. Positive values represent genes, which are expressed in increased amounts in the presence of Ciglitazone. Negative values represent genes, which are expressed in decreased amounts in the presence of Ciglitazone.

Serum IgE ELISA

Sera from the indicated animals were diluted 1:10 and analyzed by ELISA for OVA specific IgE. In brief, ELISA plates were coated with OVA overnight, washed, and blocked with BSA. Diluted serum from each animal was then added to the coated wells and washed and then biotinylated anti-mouse IgE antibodies were added. The plates were then washed, the IgE was detected using streptavidin-HRP, and the results were analyzed by spectrophotometer.

Statistical analysis. The data were analyzed by ANOVA, with treatment as a between subject factor. Fisher's PLSD post-hoc analysis was used to determine significance. The level of significance was set at P<0.05. Data were analyzed using StatView (SAS Institute Inc., Cary, N.C.).

In Vitro Assys

The compounds to be tested are incubated with T cells in vitro. The T cells are then stimulated with either antigen along with antigen presenting cells or alternatively antibodies against the T cell receptor and CD28. After 3-4 days, supernatants from these cultures can be analyzed for the presence of T_{H}2 type cytokines. Compounds that inhibit the production of T_{H}2 type cytokines would be predicted to have some effect in treating asthma, allergy and type 1 hypersensitivity. See, for example, Mueller et al., 2003; DeGrazia et al., 2003; and, Yu et al., Biochim. Biophys. Acta. 1581: 89.

Results

Clark et al. (J. Immunol. 164: 1364 (2000)) have recently demonstrated the presence of PPAR-γ in T cells, and previous studies have suggested a role for ligands of PPAR-γ in regulation of T cell cytokine production. The present inventors decided to test if T cells are direct targets of PPAR-γ ligands. Purified CD4+T cells carrying the transgenic TcR for OVA (DO11.10) were cultured in vitro with OVA and mitomycin C treated splenocytes under neutral conditions (THO priming) or with rIL-4 and anti-IFN-γ antibodies (T_{H}2 priming), or with rIL-12 (T_{H}1 priming). Following 4 days of initial culture, cells were isolated and expanded in rIL-2. These expanded antigen specific T cells were stimulated in the presence or absence of PJG2, a PPAR-γ ligand and cells analyzed for proliferation and cytokine synthesis (Table 1). As previously characterized, cells initially stimulated under T_{H}1 conditions (T_{H}1 cells) secreted more IFN-γ and IL-2 and less IL-4 and IL-5 than those initially stimulated under T_{H}2 conditions (T_{H}2 cells). The T_{H}1 and T_{H}2 cells were greater than 95% CD4+and carried the transgenic TcR (data not shown). The PPAR-γ ligand PGJ2 regulated cytokine production in both T_{H}1 and T_{H}2 cells. PGJ2 inhibited IL-4 and IL-5 production in T_{H}1 cells and inhibited IFN-γ, IL-2, and IL-5 production in T_{H}2 cells (Table 1). These data indicate that both T_{H}1 and T_{H}2 cells are direct targets of the PPAR-γ ligand PGJ2.

As PGJ2 inhibited T cell secretion of IL-5 most consistently, the effect of a synthetic ligand for PPAR-γ (Ciglitazone) was tested in a murine model of a disease that has been characterized as dependent in part on IL-5, allergic asthma (Foster et al. 1996. Exp. Med. 183:195). In this model, mice are initially exposed to OVA in complex with Alum intraperitoneally, then challenged intranasally with OVA. This protocol usually leads to increased inflammatory cell infiltration into the lung, thickening of the epithelial cells lining the bronchioles of the lung, mucous secretion and in most cases, increased presence of circulating serum IgE. These symptoms highlight the predominant T_{H}2 nature of this disease. Mice (Balb/c) were primed and with OVA intranasally as described in the Materials and Methods section. Twenty-four hours after the final intranasal challenge, mice were analyzed for immunological symptoms of allergic asthma. Controls include mice that were either unprimed (no OVA/Alum intraperitoneal injection) but challenged intranasal, or were primed and intranasally challenged (See, in addition, Mueller et al. 2003. Archives of Biochemistry and Biophysics, available on line at www.sciencedirect.com).

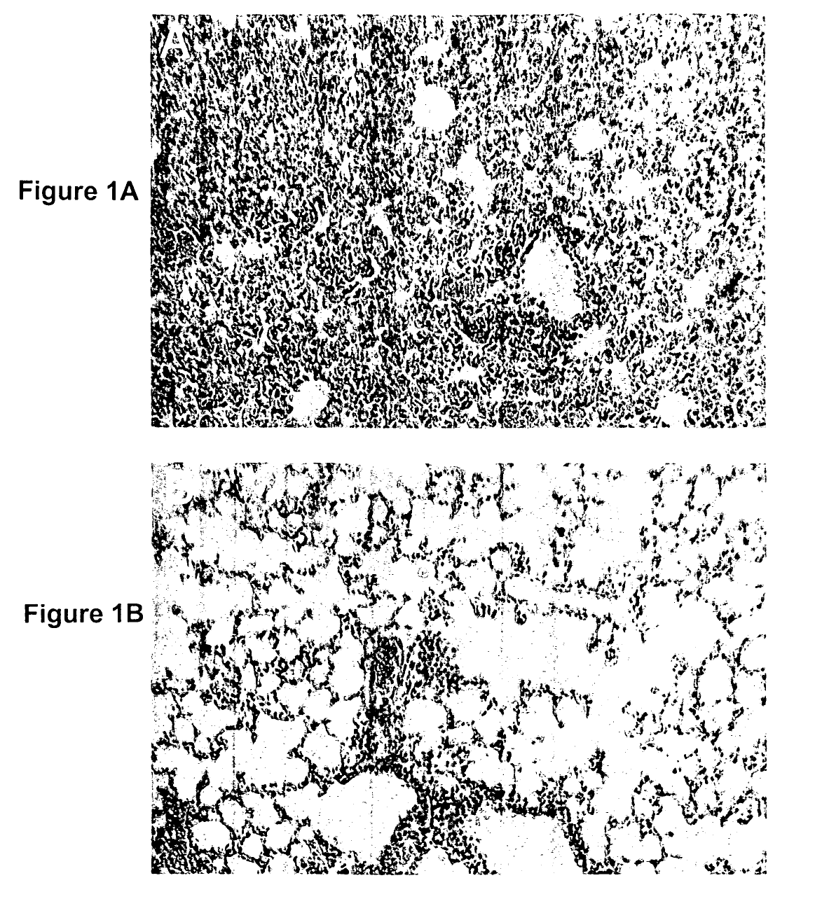

Lungs from these animals were fixed and stained with H&E to determine the inflammatory cell score as described above. A representative section is shown in FIG. 1. Lungs from mice that were either unprimed or primed, then challenged developed pathological symptoms of allergic asthma (FIG. 1A). This included increased inflammation on the bronchioles and thickening of the epithelial cells lining the lung. Treatment of mice prior to and during the course of the experiment with Ciglitazone (0.12 μM Ciglitazone/daily per mouse) significantly reduced the immunological symptoms observed, including reduced inflammation, reduced number of inflammatory foci and reduced number of granulomas (FIG. 1, Table 2). This occurred whether the mice had been primed then challenged (average histopathology score 3.0+/−0.7 vs 1+/−0.3, p=0.0040), or only challenged intranasally (average histopathology score 3.5+/−0.4 vs 0.8+/−0.2, p=0.0002, Table 2). These data suggest that treatment with Ciglatazone, the PPARγ ligand, can significantly alleviate the symptoms of allergic asthma in these mice.

TABLE 1
The PPAR-γ ligand PGJ2 inhibits Th cell cytokine production.

| Th1 | IFN-γ (pg/ml) | IL-2(pg/ml) | IL-4 (pg/ml) | IL-5 (pg/ml) |
|---|---|---|---|---|
| Control | 14698 ± 437¹ | 502 ± 5 | 191 ± 77 | 202 ± 99 |
| PGJ2 | 14370 ± 250 | 512 ± 1 | 74 ± 7* | 68 ± 13* |
| 10 mM | | | | |
| CLA | 14689 ± 370 | 522 ± 20 | ND | 150 ± 6 |
| 5 mM | | | | |
| Th2 | | | | |
| Control | 6697 ± 45 | 226 ± 49 | 1681 ± 330 | 277 ± 28 |
| PGJ2 | 3178 ± 108* | 177 ± 0* | 1861 ± 28 | 178 ± 4* |
| 10 mM | | | | |
| CLA | 5592 ± 306 | 224 ± 4 | 1863 ± 62 | 240 ± 52 |
| 5 mM | | | | |

¹Values are the mean ± SD of triplicate wells. One representative experiment of three.
ND—not done.

In human asthma, it is difficult to determine who will develop the disease until the symptoms are well underway. In order to try to mimic treatment of already developing disease, the mice were primed and then started on Ciglitazone treatment just prior to intranasal challenge with OVA (late Ciglitazone, Table 2). Late treatment with Ciglitazone significantly reduced the symptoms of allergic asthma in these mice (average histopathology score 3.0+/−0.7 vs 1.5+/−0.3, p=0.0195, Table 2). Together these data show that treatment with Ciglitazone can significantly alleviate the symptoms of allergic asthma in these mice even following disease initiation.

TABLE 2
Asthma severity is less in Ciglitazone treated mice.

| TREATMENT | UNPRIMED | PRIMED |
|---|---|---|
| Control | 3.5 ± 0.4¹ | 3.0 ± 0.7 |
| Ciglitazone | 0.8 ± 0.2* | 1.0 ± 0.3* |
| Late Ciglitazone | ND | 1.5 ± 0.3* |

¹Values are the mean ± the SE of 6 mice.
ND—not done.
*Values from Ciglitazone treated mice were significantly lower then their respective controls.

As this model of allergic asthma is driven by T cell activation and cytokine secretion, the present inventors analyzed the effect of Ciglitazone treatment on the response of splenic T cells with regards to cytokine production and proliferation (Table 3). Splenocytes from mice treated as described above were incubated in vitro with OVA in the absence of further Ciglitazone treatment and cytokine production and proliferation determined.

Table 3 illustrates that Ciglitazone treatment also significantly changed the antigen specific cytokine production profile in the splenocytes from these mice. There was a significant reduction in the secretion of IL-2, IFN-γ, and IL-4 in mice fed Ciglitazone regardless of priming (Table 3). Interestingly, while late Ciglitazone treatment significantly reduced the immunological symptoms in the lung, it only significantly reduced antigen specific IL-2 production (Table 3). Unprimed mice fed Ciglitazone also showed a trend towards a decrease in IL-5 (P=0.07) and a significant decrease in antigen specific proliferation. Thus the effect of Ciglitazone treatment on this model of allergic asthma was associated with the decreased production of IFN-γ, IL-2, and IL-4 in splenocytes.

Taken together, these data show a role for PPARγ ligands in modulating the severity of allergic asthmatic symptoms, most likely at the level of reduced cytokine production by T cells.

TABLE 3
Antigen specific immune responses were inhibited by in vivo
Ciglitazone treatment.

| | IFN-γ | IL-2 | IL-4 | IL-5 | Proliferation |
|---|---|---|---|---|---|
| | (pg/ml) | (pg/ml) | (pg/ml) | (pg/ml) | (CPM) |
| Unprimed Control | 3775 ± 199 | 264 ± 16¹ | 4 ± 1 | 36 ± 20 | 14102 ± 1430 |
| Unprimed Ciglitazone | 1837 ± 591* | 26 ± 12* | 1 ± 0* | 0 ± 0 | 5806 ± 707* |
| Primed Control | 4031 ± 215 | 56 ± 17 | 463 ± 103 | 989 ± 9 | 15820 ± 3248 |
| Primed Ciglitazone | 1996 ± 393* | 4 ± 1* | 95 ± 37* | 903 ± 87 | 10649 ± 492 |
| Primed | 4063 ± 170 | 9 ± 5* | 395 ± 92 | 875 ± 79 | 10579 ± 1477 |
| Late Ciglitazone | | | | | |

¹Values are mean ± SEM of 6 individual mice.
*Significantly lower then respective control.

PPARγ Targets in the Lung of Mice with Experimental Asthma

Microarray analysis of lungs from control and Ciglitazone treated mice following allergic asthma induction was performed. Microarray analysis of Ciglitazone treated lungs from unprimed mice (asthma severity 0.8±0.2, Table 2) vs. lungs from control treated unprimed mice (asthma severity 3.5±0.4, Table 2) revealed that expression of many genes was suppressed by Ciglitazone treatment, while only a few genes were upregulated (Table 4). We found reduced expression of several genes previously shown to regulate allergic asthma including: prostaglandin D2 (PGD2) synthase message (2.2-fold reduction) and chemokine receptor CCR4 (2.1-fold reduction; Table 4). We also found several candidate genes whose message was reduced by Ciglitazone treatment in the lung and based on their function and expression might regulate the development of allergic asthma. The message for the toll-like receptor MD1/RP105 (Ly78) and prostaglandin E2 receptor (EP2) was reduced by 2-fold in Ciglitazone treated animals (Table 4). Of the few genes that were upregulated by Ciglitazone treatment, the PigR A4 receptor was of interest and increased 2.20-fold in ciglitazone treated mice (Table 4).

Discussion

PPARγ is expressed in T cells (Clark et al. 2000) and ligands for this receptor regulate the expression of specific genes. We have demonstrated that T cells are direct targets of the PPARγ ligand PGJ₂. Both T_{H}1 and T_{H}2 cell cytokine secretion was inhibited by PGJ₂. Interestingly, IFN-γ production was inhibited by PGJ₂ but only the T_{H}2 cells and IL-4 secretion was inhibited by PGJ₂ but only in the T_{H}1 cells. IL-5 secretion was inhibited in both T_{H}1 and T_{H}2 cells. The finding that both T_{H}1 and T_{H}2 cells are direct targets of PGJ₂ is consistent with the data that show PPARγ inhibition of T_{H}1 driven autoimmune diseases as well as our data here showing PPARγ inhibition of T_{H}2 driven allergic asthma.

IL-5 is very potent at recruiting eosinophils to the lung. We therefore tested the effect of a well-established ligand for PPARγ, Ciglitazone, on a disease that has a large component of symptoms dependent on the recruitment of eosinophils. We show that Ciglitazone was extremely effective at inhibiting experimentally induced allergic asthma. This included reduced inflammatory cell infiltration as well as reduced thickening of the epithelial cells lining the bronchioles of the lung. These pathological symptoms are dependent on T_{H}2 type cytokines IL-4, IL-5, and IL-13. IL-5 production in allergic asthma is linked to the eosinophilia that occurs. Conversely, IL-13 regulation in allergic asthma seems to be independent of eosinophil recruitment but has been linked to mucous production. Thus, the ability of PPARγ ligands to reduce T cell mediated production of IL-5 may have direct effect on this disease by reducing the recruitment of eosinophils and therefor some of the inflammatory components of the disease. While mucous production was reduced by Ciglitazone treatment, the reduction was not significant, suggesting perhaps that IL-13 may not be regulated by Ciglitazone treatment to a great extent.

Of interest are the results of cytokine production from the in vitro restimulation of splenocytes from ciglitazone treated mice. We note that while some cytokines were reduced, notably, IL-5 was not affected. This could result from a combination of the effect of Ciglitazone on antigen presenting cells such as macrophages that may alter their ability or capacity to present antigen and affect the differentiation of T cells and their resulting cytokine production profiles, as well as having a direct effect on T cells. A more in-depth examination of the regulation of the immune response by these PPAR-γ ligands is necessary for differentiation between these possibilities.

The microarray analysis demonstrated that a number of genes implicated in regulating allergic asthma are downregulated by Ciglitazone in the lung. Prostaglandin D2 (PGD2) synthase message was reduced 2.2-fold compared to non-treated animals (Table 4). A role for prostaglandin D2 synthase in regulating allergic asthma has been illustrated in PGD2 synthase transgenic animals that develop increased eosinophilic inflammation. We also observed reduced expression of the chemokine receptor CCR4 (2.1-fold reduction) (Table 4). CCR4 and CCR8 have been found to be preferentially expressed on airway T cells from allergen-challenged atopic asthmatics. In addition, CCR4 knockout animals exhibit attenuated AHR in this model of allergic asthma.

We also found several candidate genes whose message was reduced by Ciglitazone treatment in the lung and based on their function and expression might regulate the development of allergic asthma. The message for the toll-like receptor MD1/RP105 (Ly78) was reduced 2-fold (Table 4). Toll receptors have been demonstrated to regulate innate immune responses in the lung and thus the nature of the response in this model of allergic asthma. Prostaglandin E2 has been reported to downregulate allergic inflammation and AHR in the lung, and we found that expression of a prostoglandin E2 receptor, EP2, was reduced (2.1 1-fold) in Ciglitazone treated animals (Table 2). Of the few genes that were upregulated by Ciglitazone treatment, the PigR A4 receptor was of interest (2.20-fold increase), as it belongs to a family of receptors that can downregulate activation of mast cells (Table 4). Suprisingly, we found little downregulation of specific cytokines for chemokines in the lung, suggesting perhaps that at this level of resolution, it will be difficult to determine whether the genes for these proteins are affected by Ciglitazone treatment in the lung.

It is possible that some of the effects of the PPAR ligands are independent of PPARγ. Indeed, PGJ₂ has been shown to covalently modify critical residues in NF-κB kinase and the DNA-binding domain of NF-κ B. The synthetic ligands of PPARγ (thiazolidenediones) may also have PPARγ-independent effects in macrophages. However, although not studied in as much detail as monocytes/macrophases, T-cells appear to respond to PGJ₂ and Ciglitazone solely in a PPARγ-dependent manner. Further experiments are necessary to determine whether all of the functions of PPARγ ligands PGJ₂ and ciglitazone on this disease are dependent on this receptor subtype.

TABLE 4
Ciglitazone regulated genes

| Name | GenBank | Description | Mean | SEM |
|---|---|---|---|---|
| Cyr61 | M32490 | Cysteine rich protein 61^{d} | 5.14 | 2.04 |
| Fos; c-fos | V00727 | c-fos^{a,d,c} | 4.82 | 1.66 |
| Enh-pending | AB016587 | Murine homolog of Rattus norvegicus | 3.37 | 0.80 |
| | | enigma^{g} | | |
| Add2 | NM_013458 | Adducin 2(β)^{d} | 3.20 | 0.75 |
| Cnn1; CN; CnnI | L49022 | h1-calponin α and β genes^{f} | 2.63 | 0.81 |
| Defb4 | AF155882 | Defensin β 4^{e} | 2.58 | 0.68 |
| Nupr1 | AF131196 | Nuclear protein 1^{g} | 2.43 | 0.48 |
| Mad4 | U32395 | Max dimerization protein 4^{c} | 2.41 | 0.48 |
| Pbef-pending | AF234625 | Pre-B-cell colony-enhancing factor^{d} | 2.38 | 0.67 |
| 2900002H16Rik | AB041584 | RIKEN cDNA 2900002H16 gene^{g} | 2.38 | 0.52 |
| Fpr1; FPR | L22181 | N-Formyl peptide chemotactic receptor^{d} | 2.28 | 0.64 |
| I11b | M15131 | Interleukin 1 β^{d} | 2.26 | 0.38 |
| Mtf1 | X71327 | Metal response element binding transcription | 2.22 | 0.28 |
| | | factor 1^{c} | | |
| Esrrg | NM_011935 | Estrogen-related receptor γ^{f} | 2.22 | 0.26 |
| Rock1 | U58512 | Rho-associated coiled-coil forming kinase | 2.21 | 0.43 |
| | | 1^{b,d} | | |
| Pira4 | U96685 | Paired-Ig-like receptor A4^{e} | 2.20 | 0.25 |
| Pkd212 | AF182033 | Polycystic kidney disease 2-like 2^{f} | 2.20 | 0.33 |
| Sgcg | AB024922 | Sarcoglycan, γ (35 kDa dystrophin- | 2.14 | 0.28 |
| | | associated glycoprotein)^{f} | | |
| Hmgcs2; mHS | U12791 | HMG CoA synthase^{b} | 2.11 | 0.27 |
| U55872 | NM_020571 | cDNA sequence U55872 | 2.05 | 0.24 |
| Ggps1 | AB016044 | Geranylgeranyl diphosphate synthase 1^{b} | 2.04 | 0.15 |
| Tnfrsf11b^{a} | AB013898 | Tumor necrosis factor receptor superfamily, | −1.89 | 0.12 |
| | | member 11b (osteoprotegerin)^{a,d} | | |
| Stat5a | U21103 | Signal transducer and activator of | −1.89 | 0.13 |
| | | transcription 5A^{c,d} | | |
| Ptn | D90225 | Pleiotrophin^{g} | −1.89 | 0.11 |
| Akt3^{a} | NM_011785 | Thymoma viral proto-oncogene 3^{a} | −1.92 | 0.10 |
| Tslpr | AF201963 | Thymic stromal-derived lymphopoietin, | −1.92 | 0.08 |
| | | receptor^{d} | | |
| Mrps12 | Y11682 | Mitochondrial ribosomal protein S12^{f} | −1.92 | 0.07 |
| Tbxas1 | NM_011539 | Thromboxane A synthase 1, platelet^{b} | −1.96 | 0.10 |
| Dlk1; FA1; ZOG; pG2; Peg9; | D16847 | SCP-1, (stromal cell derived protein-1)^{d} | −1.96 | 0.10 |
| SCP1 | | | | |
| 2410004C24Rik | AJ131957 | RIKEN cDNA 2410004C24 gene^{g} | −1.96 | 0.04 |
| Bglap1;OG1; mOC-A | L24431 | Osteocalcin^{f} | −1.96 | 0.06 |
| Myod1 | M84918 | Myogenic differentiation 1^{c} | −2.00 | 0.07 |
| Ly78 | D37797 | Lymphocyte antigen 78^{e} | −2.00 | 0.08 |
| Mx2 | AB029920 | Myxovirus (influenza virus) resistance 2^{g} | −2.00 | 0.09 |
| Jtb | AB016490 | Jumping translocation breakpoint^{g} | −2.00 | 0.09 |
| Rmp-pending | AF091096 | RPB5-mediating protein^{g} | −2.00 | 0.10 |
| Brp14 | X61450 | Brain protein 14^{g} | −2.00 | 0.05 |
| Fyb | AF061744 | FYN binding protein^{d} | −2.00 | 0.09 |
| Scg2; Chgc; SgII | X68837 | Secretogranin II^{f} | −2.00 | 0.12 |
| Ereg | D30782 | Epiregulin^{d} | −2.04 | 0.09 |
| Krt2-6g | AB033744 | Keratin complex 2, basic, gene 6g^{f} | −2.04 | 0.09 |
| Ppy | M18208 | Pancreatic polypeptide^{g} | −2.04 | 0.07 |
| Cmkbr4 | U15208 | Chemokine (C—C) receptor4^{d} | −2.04 | 0.07 |
| Slc10a2 | AB002693 | Solute carrier family 10, member 2^{f} | −2.08 | 0.11 |
| Madh5 | AF063006 | Murine homolog of Drosophila MAD 5^{g} | −2.08 | 0.12 |
| Siat1 | D16106 | Sialyltransferase 1 (β-galactoside | −2.13 | 0.08 |
| | | α-2,6-sialyltransferase)^{b} | | |
| Ephb6 | L77867 | Eph receptor B6^{d} | −2.13 | 0.10 |
| Dnajb3 | U95607 | DnaJ (Hsp40) homolog subfamily B, | −2.13 | 0.06 |
| | | member 3^{f} | | |
| Drd4; D4R; Drd-4 | U19880 | D4 dopamine receptor^{d} | −2.13 | 0.00 |
| alpha-Alb | AJ011080 | Afamin precursor^{g} | −2.13 | 0.08 |
| Ptgds; PGD2; Ptgs3; L-PGDS | Y10138 | Prostaglandin D synthase, putative^{b} | −2.17 | 0.12 |
| Ndufa1 | Y07708 | NADH dehydrogenase (ubiquinone) 1 α | −2.17 | 0.15 |
| | | subcomplex, 1^{b} | | |
| Myf6 | X59060 | Myogenic factor 6^{c} | −2.17 | 0.12 |
| Npy5r | AB001346 | Neuropeptide Y receptor Y5^{d} | −2.17 | 0.12 |
| | AB007696 | Prostaglandin E receptor subtype EP2^{d} | −2.17 | 0.11 |
| Klk26 | K01831 | Kallikrein 26^{b,d} | −2.22 | 0.09 |
| Six2 | D83147 | Murine homolog of Drosophila sine | −2.22 | 0.00 |
| | | oculis-related homeobox 2^{c} | | |
| Pparbp | AF000294 | Peroxisome proliferator activated receptor | −2.22 | 0.11 |
| | | binding protein^{c} | | |
| Cyp7b1 | U36993 | Cytochrome P450, 7b1^{b} | −2.27 | 0.13 |
| Hils1 | AB022320 | Histone H1-like protein in spermatids 1^{c} | −2.27 | 0.11 |
| Cops7b | AF071317 | Murine homolog of Arabidopsis thaliana | −2.27 | 0.09 |
| | | COP9 (constitutive photomorphogenic)^{d} | | |
| Pcdh10 | NM_011043 | Protocadherin 10^{f} | −2.27 | 0.10 |
| Slc30a4 | AF003747 | Solute carrier family 30 (zinc transporter), | −2.27 | 0.10 |
| | | member 4^{f} | | |
| Il12b | M86671 | Interleukin 12b^{d} | −2.27 | 0.11 |
| Stac | D86639 | Src homology three (SH3) and cysteine rich | −2.27 | 0.09 |
| | | domain^{d} | | |
| Rrm2; R2 | X15666 | M2 subunit of mouse ribonucleotide | −2.32 | 0.08 |
| | | reductase (EC 1.17.4.1)^{b} | | |
| Nrg4 | AF083067 | Neuregulin 4^{d} | −2.32 | 0.12 |
| Acr | D00754 | Preproacrosin^{b} | −2.38 | 0.07 |
| 6530404A22Rik | AF194970 | RIKEN cDNA 6530404A22 gene^{g} | −2.38 | 0.12 |
| Mpg; Aag; APNG; Mid1; | X74509 | N-Methylpurine-DNA glycosirase^{b} | −2.43 | 0.13 |
| Prl | X04418 | Prolactin^{d} | −2.43 | 0.06 |
| Cyp2c39 | AF047726 | Cytochrome P450, 2c39^{b} | −2.44 | 0.08 |
| Mpg; Aag; APNG; Mid1; | X74509 | N-Methylpurine-DNA glycosirase^{c,b} | −2.44 | 0.13 |
| AI326268 | | | | |
| Ar | X59411 | Androgen receptor^{c} | −2.44 | 0.07 |
| Prl | X04418 | Prolactin^{d} | −2.44 | 0.06 |
| Slc22a2 | AJ006036 | Solute carrier family 22 (organic cation | −2.44 | 0.07 |
| | | transporter), member 2^{f} | | |
| Krtap14 | AF003691 | Keratin associated protein 14^{f} | −2.44 | 0.03 |
| Tssc3 | Y15443 | Tumor-suppressing subchromosomal | −2.50 | 0.10 |
| | | transferable fragment 3^{a} | | |
| Nppb; BNP; AA408272 | S58667 | Type-B natriuretic peptide^{d} | −2.5 | 0.09 |
| Xtrp3 | AF075261 | X transporter protein 3^{f} | −2.50 | 0.11 |
| Pcdh8 | AF231125 | Protocadherin 8^{f} | −2.50 | 0.10 |
| H2-M9 | AF016308 | MHC class Ib antigen (M9)^{e} | −2.50 | 0.08 |
| Cyp2c29 | D17674 | Cytochrome P450, 2c29^{b} | −2.63 | 0.10 |
| Csk | U05247 | c-src tyrosine kinase^{b, d} | −2.63 | 0.08 |
| Zic1 | D32167 | Zinc finger protein of the cerebellum 1^{c} | −2.63 | 0.09 |
| Slc23a2 | AF058318 | Solute carrier family 23 (nucleobase | −2.63 | 0.12 |
| | | transporters), member 2^{f} | | |
| Ors6 | AF121974 | G-protein-coupled receptor; odorant | −2.63 | 0.07 |
| | | receptor S6^{d} | | |
| Zic3 | D70849 | Zinc finger protein of the cerebellum 3^{c} | −2.70 | 0.09 |
| Pcdha5 | AB008181 | Protocadherin α 5^{f} | −2.70 | 0.10 |
| Ubl1 | AF033353 | Ubiquitin-like 1^{f} | −2.70 | 0.11 |
| Cd53 | X97227 | CD53 antigen^{e} | −2.70 | 0.08 |
| | NM_010869 | | −2.70 | 0.06 |
| Fads2 | AF126798 | Fatty acid desaturase 2^{b} | −2.78 | 0.11 |
| Oaz3; AZ-3; Oaz-t | AB016275 | Oaz-t^{b} | −2.78 | 0.10 |
| Gad1 | Z49976 | Glutamic acid decarboxylase 1^{b} | −2.78 | 0.12 |
| Hoxa7; Hox-1.1 | U15972 | Hoxa7^{c} | −2.78 | 0.05 |
| Nh1h1 | M82874 | Nescient helix-loop-helix 1^{c} | −2.78 | 0.08 |
| Hoxa10 | L08757 | Homeo box A10^{c} | −2.78 | 0.01 |
| Fgf6 | M92416 | Fibroblast growth factor6^{d} | −2.78 | 0.00 |
| Mb12 | D11440 | Mannose binding lectin, serum (C)^{e} | −2.78 | 0.09 |
| Defcr-rs2 | M33227 | Defensin related cryptdin, related sequence 2^{g} | −2.78 | 0.09 |
| Setdb1 | AF091628 | SET domain, bifurcated 1^{g} | −2.78 | 0.08 |
| Snfllk | NM_010831 | SNF1-like kinase^{d} | −2.78 | 0.10 |
| Sdc1; Synd; CD138; syn-1 | Z22532 | Mus musculus syndecan-1^{f}. | −2.78 | 0.09 |
| Defcr5 | U12560 | Mus musculus 129 cryptdin 5 gene, | −2.86 | 0.12 |
| | | complete cds.^{f} | | |
| Siah2 | Z19581 | Seven in absentia 2^{f} | −2.86 | 0.06 |
| Bcl2l11 | AF032459 | BCL2-like 11 (apoptosis facilitator)^{a} | −2.9 | 0.08 |
| Uncx4.1 | AJ001116 | Murine homolog of C. elegans Unc4.1 homeobox^{c} | −2.94 | 0.05 |
| M32460 | M32460 | Histone H3 (H3.1-221)^{c} | −3.03 | 0.08 |
| T | X51683 | Brachyury^{c} | −3.15 | 0.06 |
| Dbn1 | AF187147 | Drebrin 1^{f} | −3.15 | 0.08 |
| Tacc2 | AB041546 | Transforming, acidic coiled-coil containing | −3.15 | 0.08 |
| | | protein 2^{g} | | |
| Gfap | M25937 | Glial fibrillary acidic protein^{f} | −3.15 | 0.08 |
| Cnr2 | X93168 | Cannabinoid receptor 2 (macrophage)^{d} | −3.15 | 0.07 |
| Spil-1 | M75721 | Serine protease inhibitor 1-1^{b} | −3.22 | 0.06 |
| Ptdss2 | AF099053 | Phosphatidylserine synthase 2^{b} | −3.33 | 0.07 |
| T2r5 | AF227147 | Mus musculus candidate taste receptor T2R5^{d} | −3.33 | 0.06 |
| | | gene, complete cds. | | |
| Dnmt2 | AF012129 | DNA methyltransferase 2^{b,c} | −3.45 | 0.07 |
| CIcl | AJ011107 | MRNA for 3′ UTR of CIcl gene^{f} | −3.45 | 0.08 |
| Edil3 | AF031525 | EGF-like repeats and discordin I-like domains | −3.57 | 0.07 |
| | | 3^{d} | | |
| Pbx1 | NM_008783 | Pre B-cell leukemia transcription factor 1^{c} | −3.85 | 0.08 |
| Napb | X61455 | N-Ethylmaleimide sensitive fusion protein | −3.85 | 0.06 |
| | | attachment protein beta^{f} | | |
| Zfp28 | M36516 | Zinc finger protein 28^{c} | −4.00 | 0.06 |
| Zfp26 | X12592 | Zinc finger protein 26^{c} | −4.00 | 0.06 |
| Egf | J00380 | Epidermal growth factor^{d} | −4.17 | 0.07 |
| Wif1 | NM_011915 | Wnt inhibitory factor 1^{f} | −4.17 | 0.07 |
| Ninj2 | AF205634 | Ninjurin 2^{f} | −4.35 | 0.05 |
| Hmga2; pg; Hmgic; pygmy; | X99915 | HMGI-C^{c} | −4.76 | 0.05 |
| HMGI-C | | | | |
| Oprs1 | AF004927 | Opioid receptor, sigma 1^{d} | −4.76 | 0.07 |
| Phxr4 | X12806 | Per-hexamer repeat gene 4^{g} | −4.76 | 0.06 |
| Schip1 | AF145716 | Schwannomin interacting protein 1^{g} | −5.26 | 0.05 |
| Mybl1 | L35261 | Myeloblastosis oncogene-like 1^{a} | −5.55 | 0.05 |
| Zp3 | M20026 | Zona pellucida glycoprotein 3^{f} | −6.67 | 0.10 |
| | U95783 | it Mus musculus endogenous provirus Imposon1 | −6.67 | 0.05 |
| | | envelope gene, partial cds, and 3′ long | | |
| | | terminal repeat, complete sequence.^{g} | | |
| Galt | M96265 | Galactose-1-phosphate uridyl transferase^{b} | −7.14 | 0.11 |
| Rax | AF001906 | Retina and anterior neural fold homeobox^{c} | −7.14 | 0.13 |
| Gcm2 | D88611 | Murine homolog of Drosophila glial cells | −7.14 | 0.10 |
| | | missing homolog 2^{c} | | |
| Mef2b | D50311 | Myocyte enhancer factor 2B^{c} | −8.33 | 0.11 |
| Ras12-9 | L32752 | RAS-like, family 2, locus 9^{b,d} | −11.11 | 0.06 |
| Rad51 | D13473 | Murine homolog of S. cerevisiae RAD51^{b,c} | −14.28 | 0.06 |
| Mif | L10613 | Macrophage migration inhibitory factor^{d} | −20.0 | 0.04 |
| Guca2b | NM_008191 | Guanylate cyclase activator 2b (retina)^{d} | −50.00 | 0.01 |
| Sema4b | X85992 | Sema domain, immunoglobulin domain (Ig), | −50.00 | 0.01 |
| | | transmembrane domain (TM), and short | | |
| | | cytoplasmic domain, (semaphorin) 4B^{f} | | |

^{a}Apoptosis, cancer, and cell cycle.
^{b}Enzymes.
^{c}Nucleic acid binding.
^{d}Signaling.
^{e}Immunity.
^{f}Other.
^{g}Unclassified.

While the invention has been particularly shown and described with reference to preferred embodiments thereof, it will be understood by those skilled in the art that various alterations in form and detail may be made therein without departing from the spirit and scope of the invention. All cited patents, patent applications, publications and other documents cited in this application are herein incorporated by reference in their entirety.

We claim:

1. A method for treating a subject having, or susceptible to having, a type I hypersensitivity, asthma or an allergy comprising administering a therapeutically effective amount of at least one PPAR-γ agonist, or derivative thereof, to said subject, wherein said administration of said at least one PPAR-γ agonist, or derivative thereof, is effective to treat said type I hypersensitivity, asthma or allergy in said subject.

2. The method of claim 1 wherein said PPAR-γ agonist is selected from the group consisting of a thiazolidinedione and a non-thiazolidinedione PPAR-γ agonist.

3. The method of claim 1 wherein said at least one PPARγ agonist is selected from the group consisting of Ciglitazone, Troglitazone, Rosiglitazone, Pioglitazone, Englitazone, RXR activator LGD1069, and prostaglandin J2.

4. The method of claim 1, wherein said subject has, or is susceptible to having, an asthma.

5. The method of claim 4, wherein said asthma is allergic asthma.

6. The method of claim 1, wherein said therapeutically effective amount of said PPAR-γ agonist is approximately from 2 mg/kg to 10 mg/kg per day.

7. The method of claim 1, wherein said therapeutically effective amount of said PPAR-γ agonist is approximately 2 mg/kg per day.

8. The method of claim 1, wherein said administering is selected from the group consisting of aerosol, parenteral, oral, intravenous, intramuscular, intraperitoneal, transdermal, rectal, buccal and subcutaneous administering.

9. The method of claim 1, wherein said subject is a mammal.

10. The method of claim 9, wherein said mammal is human.

11. The method of claim 2, wherein said PPAR-γ agonist is a thiazolidinedione derivative.

12. The method of claim 11, wherein said thiazolidinedione derivative is administered by a route selected from the group consisting of aerosol, parenteral, oral, intravenous, intramuscular, intraperitoneal, transdermal, rectal, buccal and subcutaneous administration.

13. The method of claim 11, wherein said thiazolidinedione derivative comprises a thiazolidinedone-2 derivative or a 4-diketone substituted derivative.

14. The method of claim 11, wherein said therapeutically effective amount of said thiazolidinedione derivative is approximately 2 mg/kg to 10 mg/kg per day.

15. The method of claim 11, wherein said therapeutically effective amount of said thiazolidinedione derivative is approximately 2 mg/kg per day.

16. The method of claim 11, wherein said subject is a mammal.

17. The method of claim 16, wherein said mammal is a human.

18. The method of claim 2, wherein said PPAR-γ agonist is a non-thiazolidinedione PPAR-γ agonist.

19. The method of claim 18, wherein said non-thiazolidinedione PPAR-γ agonist. is administered by a route selected from the group consisting of aerosol, parenteral, oral, intravenous, intramuscular, intraperitoneal, transdermal, rectal, buccal and subcutaneous administration.

20. The method of claim 18, wherein said non-thiazolidinedione PPAR-γ agonist comprises a piperazine or heterocycle derivative.

21. The method of claim 18, wherein said therapeutically effective amount of said non-thiazolidinedione PPAR-γ agonist is approximately 2 mg/kg to 10 mg/kg per day.

22. The method of claim 18, wherein said therapeutically effective amount of said non-thiazolidinedione PPAR-γ agonist is approximately 2 mg/kg per day.

23. The method of claim 18, wherein said subject is a mammal.

24. The method of claim 23, wherein said mammal is a human.

25. A method for treating a subject having, or susceptible to having, a type I hypersensitivity, asthma or allergy, comprising administering to said subject a therapeutically effective amount of a compound comprising Formula I: 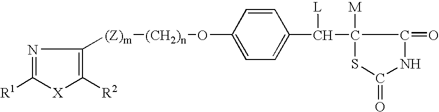
wherein R¹ is hydrogen, hydrocarbon residue, or heterocyclic residue which may each be substituted;
R² is hydrogen or lower alkyl which may be substituted by a hydroxyl group;
X is an oxygen or sulfur atom;
Z is a hydroxylated methylene or carbonyl;
m is a value of 0 or 1;
n is an integer having a value of from 1 to 3; and
L and M combine with each other and cooperate jointly to form a linkage and a plurality of salts.

26. The method of claim 25, wherein said subject has, or is susceptible to having, an asthma.

27. The method of claim 26, wherein said asthma is allergic asthma.

28. The method of claim 25, wherein said therapeutically effective amount of said compound is approximately from 2 mg/kg to 10 mg/kg per day.

29. The method of claim 25, wherein said therapeutically effective amount of said PPAR-γ agonist is approximately 2 mg/kg per day.

30. The method of claim 25, wherein said administering is selected from the group consisting of aerosol, parenteral, oral, intravenous, intramuscular, intraperitoneal, transdermal, rectal, buccal, or subcutaneous administration.

31. The method of claim 25, wherein said subject is a mammal.

32. The method of claim 31, wherein said mammal is a human.

33. An in vivo method of identifying a compound effective to treat type I hypersensitivity, asthma or allergy in a subject comprising:
a) contacting a group of one or more subjects with a test compound to form a first population;
b) contacting a different group of one or more subjects with a PPAR-γ agonist to form a second population;
c) inducing type I hypersensitivity, asthma or said allergy in said first and second populations; and,
d) comparing one or more symptoms of said type I hypersensitivity, asthma or allergy in said first and second populations;
wherein when said one or more symptoms of said type I hypersensitivity, asthma or allergy in said first population is less than or the same as said one or more symptoms of said type I hypersensitivity, asthma or allergy in said second population, a compound effective to treat type I hypersensitivity, asthma or allergy in a subject is identified.

34. The method of claim 33, wherein said one or more symptoms is selected from the group consisting of an increase in T_{H}2 type cytokines, lung airway inflammation, eosinophil infiltration, mucous production in the lung, airway hyperreactivity (AHR) and elevated serum IgE levels.

35. The method of claim 33, wherein said subject is a mammal.

36. The method of claim 35, wherein said mammal is human.

37. The method of claim 33, wherein said asthma is allergic asthma.

38. A compound identified by the method of claim 33.

39. The compound of claim 38 in a pharmaceutically acceptable carrier.

40. The method of claim 33, wherein said agonist is Ciglitazone.

41. A method of regulating T_{H}2 cell function in the lung airway of a subject in need of said regulating comprising administering to said subject an amount of a PPAR-γ agonist effective to regulate said T_{H}2 cell function in said lung airway of said subject.

42. The method of claim 41, wherein said T_{H}2 cell function is selected from the group consisting of T_{H}2 cell cytokine production, inflammation, eosinophil infiltration, mucous production, airway hyperreactivity and epithelial cell thickening.

43. The method of claim 42, wherein said T_{H}2 cell cytokine production comprises production of IL-4, IL-5 and IL-13.

44. An in vitro method for identifying a compound effective to treat type I hypersensitivity, asthma or allergy in a subject comprising:
a) culturing a first T cell population under T_{H}2 priming conditions to obtain a primed first cell population;
b) culturing a second T cell population under T_{H}2 priming conditions to obtain a primed second cell population;
c) stimulating said first primed cell population with a PPARγ agonist;
d) stimulating said second primed cell population with said test compound; and,
e) comparing the amount of secretion of one or more cytokines from said cell populations in part c) and part d);
wherein when the cytokine secretion from said cell population of part d) is less than or equal to the cytokine secretion from the cell population of part c), a compound effective to treat type I hypersensitivity, asthma or allergy in a subject is identified.

45. The method of claim 44, wherein said PPARγ agonist is Ciglitazone.

46. The method of claim 44, wherein said one or more cytokines is selected from the group consisting of IL-2, IL-5, IL-13 and IFNγ.

47. The method of claim 44, wherein said subject is a mammal.

48. The method of claim 47, wherein said mammal is human.

49. A compound identified by the method of claim 44.
